(19) United States
(12) Patent Application Publication
(10) Pub. No.: US 2015/0127080 A1
(43) Pub. Date: May 7, 2015

(54) MEDICAL IMPLANTABLE LEAD AND MANUFACTURE THEREOF

(71) Applicant: COMPOSITE MATERIALS TECHNOLOGY, INC., Shrewsbury, MA (US)

(72) Inventor: James Wong, Shrewsbury, MA (US)

(21) Appl. No.: 14/517,312

(22) Filed: Oct. 17, 2014

Related U.S. Application Data
(60) Provisional application No. 61/898,988, filed on Nov. 1, 2013.

(51) Int. Cl. A61N 1/05; A61N 1/04

(52) U.S. Cl. CPC A61N 1/0551; A61N 1/0492
USPC 607/116; 607/152; 29/825

(57) ABSTRACT

A medical implantable lead includes a core formed of elongated filaments formed of a first biocompatible conductive wire in a matrix formed of a second biocompatible metal, surrounded by a biomechanical insulating material, wherein filaments of the first biocompatible conductive wire extend from one or both ends of the lead.

### CROSS REFERENCE TO RELATED APPLICATIONS

This application claims priority from U.S. Provisional Application Ser. No. 61/898,988, filed Nov. 1, 2013, the contents of which are incorporated herein by reference in their entirety.

### FIELD OF THE INVENTION

The present invention relates to systems and methods for treating spinal cord injuries and other pathologies, and to medical electrode leads and methods for manufacture thereof for use in systems and methods for treating spinal cord injuries and other pathologies. The invention has particular utility in connection with medical implantable leads as replacements or patches for damaged nerves and will be described in connection with such utility, although other utilities are contemplated.

### BACKGROUND OF THE INVENTION

Severe spinal trauma, i.e., in which nerves are severed, and other pathologies such as spina bifida, spinal cord tumors, cauda equina syndrome and the like has left many individuals paralyzed or partially paralyzed, as well as loss of bodily functions (bladder/intestinal/sexual). Paralysis occurs when spinal cord nerves are severed. Generally when the spinal cord is fractured and nerves severed, the patient will lose all use of muscles below the spinal cord fracture.

Researchers have proposed re-growing severed nerves using among others, such as stem cell therapy; however, while such attempts show promise, to date such attempts have not been successful.

The intensity of electrical nerve signals, i.e. signals from the brain to the muscles are extremely low. Thus, the use of conventional small gauge wires to reconnect the severed ends of spinal nerves, or to create new connections between the brain and isolated muscle groups or organs is extremely difficult.

### SUMMARY OF THE INVENTION

The present invention overcomes the aforesaid and other problems with the prior art, by providing an extremely high surface area fibrous bundle, formed of extremely fine gauge (2 to 50 um diameter fibers) electrically conductive biocompatible metal as replacements or patches for damaged nerves.

The preferred metal comprises tantalum, although other valve metals such as niobium, titanium, zirconium and its alloys which are also biocompatible, advantageously may be used in accordance with the present invention.

The fibrous bundle is formed by combining shaped elements of, e.g. tantalum, or another biocompatible metal, such as niobium, tantalum or titanium, with a ductile material such as copper or silver to form a billet, The billet is then sealed in an extrusion can, and extruded and drawn following the teachings of my prior PCT Application Nos. PCT/US07/79249 or PCT/US08/86460, or my prior U.S. Pat. Nos. 7,480,978 or 7,146,709. The drawn wire is then wrapped or coated in an electrically insulating layer or sheath, leaving one or both ends exposed, and the exposed end or ends are etched, e.g., in HNO₃—H₂O to completely remove all the copper or silver, surrounding the exposed end or ends, leaving the extremely fine fibers of tantalum extending from the bundle.

### BRIEF DESCRIPTION OF THE DRAWINGS

Further features and advantages of the present invention will be seen from the following detailed description, taken in conjunction with the accompanying drawings, wherein:

FIG. 1 diagrammatically illustrates the overall process for producing a medical implantable lead in accordance with the present invention;

FIG. 2 shows an insulated, wrapped bundle in accordance with the present invention; and

FIG. 3 illustrates an implantable system in accordance with the present invention.

### DETAILED DESCRIPTION

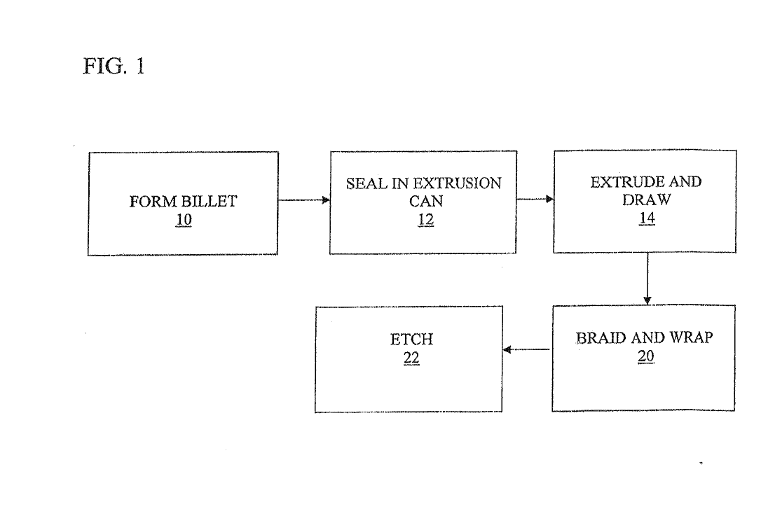

Referring to FIG. 1, the process starts with the fabrication of valve metal filaments, such as tantalum, by combining shaped elements of tantalum with a ductile material, such as copper or silver to form a billet at step 10. Copper is an essential trace mineral that is important for both physical and mental health. However, since excess copper has been associated with certain health problems, silver is preferred. Silver also has the advantage of being anti-bacterial and anti-microbial in the body. The billet is then sealed in an extrusion can in step 12, and extruded and drawn in step 14 following the teachings of my prior PCT Applications No. PCT/US07/79249 or PCT/US08/86460, or my prior U.S. Pat. Nos. 7,480,978 or 7,146,709 to reduce the tantalum filaments to 1 to 50 μm diameter, preferably 10 to 25 microns, more preferably 1-10 microns. Filaments having a round cross section are preferred from a standpoint of flexibility, although filaments having other cross-sectional shapes can also advantageously be formed.

The resulting drawn wire is then wrapped in an insulating envelope, 18 in a wrapping step 20, leaving one or both ends exposed, and the copper or silver is then removed from the end or ends, e.g. by etching in, e.g. nitric acid in an etching step 22.

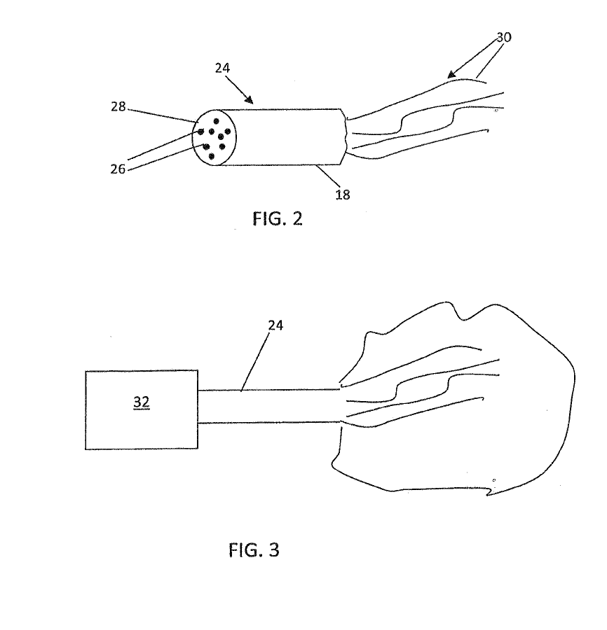

Referring to FIG. 2, the resulting cable comprises a bundle 24 of extremely fine, highly flexible, ductile tantalum fibers 26, in a copper or silver matrix 28, and surrounded by insulation 18, except at the end or ends where the highly flexible, ductile tantalum fibers are exposed.

The resulting product comprises a bundle 24 of highly flexible, ductile, fine gauge (5 to 50 μm) metallic filaments supported within an insulation wrapped metal core 26 with exposed filaments 30 at one or both ends. A feature and advantage of the present invention is that each filament is bonded to a conductive metal. In essence, each filament is an electrode bonded to a metal post. The exposed filament end or ends of the wire provide an extremely fine high surface area and as such can significantly increase the conductivity of the extremely small electrical signals encountered in the body. Moreover, being formed of a biocompatible material, the exposed filament end or ends advantageously may promote soft tissue growth and in time become one with nerves and muscles. Also, if desired, electrical pulses may be transmitted through the filaments to stimulate nerve growth.

Referring to FIG. 3, the resulting bundle may then be implanted into the body and the ends attached to the respective ends of a severed nerve or nerves. Alternatively, one end of the bundle may be attached to the nerves using conventional medical techniques, and the other end connected to a spinal cord stimulator 32 or the like following the teachings of US Published Application US 2012/0330391 or as described in the recently published article by Gorm Palmgren “Shocking the Spine Back to Life, Science Illustrated, November/December 2012, pages 44-47, or to a prosthetic limb such as described in the Journal Science Translational Medicine, abstract in the New York Times, Oct. 14, 2014.

While the present invention has particular utility in connection with medical electrode leads as replacements for damaged nerves, the invention also advantageously may be used in connection with other pathologies including, for example, implantable leads for pacemakers and defibrillators, for pain management and other devices implanted into the body, or employed as “electronic tattoos” as a patch adhered to the skin or other tissue as described in the recently published article by Nanshu Lu in Technology Review, September/October, 2012, page 64.

The invention claimed is:

1. A medical lead comprising a core formed of elongated filaments formed of a first biocompatible conductive wire in a matrix formed of a second biocompatible metal, surrounded by a biomechanical insulating material, wherein filaments of the first biocompatible conductive wire extend from one or both ends of the lead.

2. The lead of claim 1, wherein the first conductive wire comprises a valve metal.

3. The lead of claim 2, wherein the valve metal is selected from the group consisting of titanium, niobium, zirconium and its alloys.

4. The lead of claim 1, wherein the second biocompatible metal comprises copper.

5. The lead of claim 1, wherein the second biocompatible metal comprises silver.

6. The lead of claim 1, wherein the filaments of the first biocompatible metal have a thickness of 1-50 microns.

7. The lead of claim 1, wherein the first biocompatible metal filaments have a thickness of 10-25 microns.

8. The lead of claim 1, wherein the first biocompatible metal filaments have a thickness of 1-10 microns.

9. A medical implantable device comprising an implantable pulse generator and a medical lead as claimed in claim 1.

10. An electronic tattoo comprising a medical lead as claimed in claim 1, in the form of a patch for adherence to the skin.

11. A method for forming an electronic tattoo for adherence to the skin or an artificial lead for implanting in living tissue of animals, which comprises steps of:
(a) sealing a billet comprised of biocompatible metal filaments and a ductile metal an extrusion can and extruding and drawing the billet;
(b) bundling the extruded and drawn filaments from step (a) in an electrically insulating layer or sheath leaving at least one end exposed; and
(c) etching the exposed end to remove the ductile material, at least in part.

12. The method of claim 11, wherein the first biocompatible metal comprises a valve metal.

13. The method of claim 12, wherein the valve metal comprises niobium, tantalum, titanium or zirconium or alloys thereof.

14. The method of claim 11, wherein the ductile metal comprises copper or silver.
